SEQUENCE LISTING (13 sequences)

SEQ ID NO: 1 - protein, 131 aa, Artificial Sequence
  Synthetic
MGWSCIILFLVATATGVHSNIVLTQSPASLAVSLGQRATISCRASESVDSYGNSFMHWYQQKPGQPPKLLIYLASNLESGVPARFSGSGSRTDFTLTIDPVEADDVATYYCQQNNGDPYTFGGGTKLEIIR

SEQ ID NO: 2 - protein, 140 aa, Artificial Sequence
  Synthetic
MGWSCIILFLVATATGVHSEVQLVESGGGLMQPKGSLKLSCAASGFTFKTYALNWVRQAPGTGLEWVARIRSKSNNYTTYYADSVKDRFTISRDDSQNMLYLQMNNLKTEDTAMYYCVRPQSGTSFAYWGQGTLVTVSAA

SEQ ID NO: 3 - protein, 126 aa, Artificial Sequence
  Synthetic
MGWSCIILFLVATATGVHSDIVMTQSQKFMSTSVGDRVSVTCKASQYVGTNVAWYQQKPGQSPKALIQSASYRCSGVPDRFTGSGSGTDFTLTISNVQSEDLADYFCQQYNTYVTFGGGTKLELKR

SEQ ID NO: 4 - protein, 141 aa, Artificial Sequence
  Synthetic
MGWSCIILFLVATATGVHSQVRLQQPGAELVRPGASVKLSCKASGYTFTTYWMNWVKQRPEDGLEWIGRIDPYDSETHYNQNFKDKAILTVDKSSSTAYMQLSSLTYEDSAVYYCTRKEGRQWGAYFDYWGQGTTLTVSSA

SEQ ID NO: 5 - protein, 106 aa, Artificial Sequence
  Synthetic
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 325 aa, Artificial Sequence
  Synthetic
STKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - DNA, 19 bp, Artificial Sequence
  Synthetic
gtttgcagcagatccaggg

SEQ ID NO: 8 - DNA, 27 bp, Artificial Sequence
  Synthetic
atacgcgtgtttgcagcagatccaggg

SEQ ID NO: 9 - DNA, 22 bp, Artificial Sequence
  Synthetic
gagttccaagtcacagtcactg

SEQ ID NO: 10 - DNA, 29 bp, Artificial Sequence
  Synthetic
atacgcgtagttgtatctccacacccagg

SEQ ID NO: 11 - DNA, 22 bp, Artificial Sequence
  Synthetic
gccatcaatcttccacttgaca

SEQ ID NO: 12 - DNA, 27 bp, Artificial Sequence
  Synthetic
atacgcgtactgaggcacctccagatg

SEQ ID NO: 13 - DNA, 26 bp, Artificial Sequence
  Synthetic
atatagatctgggggggggggggggg

(19) United States
(12) Patent Application Publication
(10) Pub. No.: US 2017/0107294 A1
(43) Pub. Date: Apr. 20, 2017

(54) ANTI-CD14 ANTIBODIES AND USES THEREOF

(71) Applicants: Nordlandssykehuset HF, Bodø (NO); Norwegian University of Science and Technology CO/ NTNU Technology Transfer AS, Trondheim (NO); University of Oslo, Blindern (NO)

(72) Inventors: Terje Espevik, Trondheim (NO); Tom Eirik Mollnes, Bodø (NO); Inger Sandlie, Oslo (NO); Corinna Lau, Bodø (NO); Kristin S. Gunnarsen, Oslo (NO)

(21) Appl. No.: 15/126,938

(22) PCT Filed: Mar. 21, 2014
(86) PCT No.: PCT/IB2014/001276

§ 371 Date: Sep. 16, 2016

(51) Int. Cl. C07K 16/28; A61K 45/06; A61K 39/395; C07K 14/765

(52) U.S. Cl. CPC C07K 16/2896; C07K 14/765; A61K 45/06; A61K 39/3955; C07K 2317/24; C07K 2317/56; C07K 2317/52; C07K 2319/31; C07K 2317/76; A61K 2039/505

(57) ABSTRACT

The present invention relates to chimeric anti-CD14 antibodies and methods of using the same. In some embodiments, the present invention relates to the use of chimieric anti-CD 14 antibodies in research, diagnostic, and therapeutic applications. In one embodiment, the anti-CD14 antibody has a variable light chain of SEQ ID NO: 1 and a variable heavy chain of SEQ ID NO: 2 (isolated from the hybridoma clone 18D11). In another embodiment, the anti-CD14 antibody has a variable light chain of SEQ ID NO: 3 and a variable heavy chain of SEQ ID NO: 4 (isolated from the hybridoma clone Mil2).

### FIELD OF THE INVENTION

The present invention relates to chimeric anti-CD14 antibodies and methods of using the same. In some embodiments, the present invention relates to the use of chimieric anti-CD14 antibodies in research, diagnostic, and therapeutic applications.

### BACKGROUND

Sepsis is a major cause of morbidity and mortality in humans and other animals. It is estimated that 400,000-500,000 episodes of sepsis resulted in 100,000-175,000 human deaths in the U.S. alone in 1991. Sepsis has become the leading cause of death in intensive care units among patients with non-traumatic illnesses. [G. W. Machiedo et al., Surg. Gyn. & Obstet. 152:757-759 (1981).] It is also the leading cause of death in young livestock, affecting 7.5-29% of neonatal calves [D. D. Morris et al., Am. J. Vet. Res. 47:2554-2565 (1986)], and is a common medical problem in neonatal foals. [A. M. Hoffman et al., J. Vet. Int. Med. 6:89-95 (1992).] Despite the major advances of the past several decades in the treatment of serious infections, the incidence and mortality due to sepsis continues to rise. [S. M. Wolff, New Eng. J. Med. 324:486-488 (1991).]

Sepsis is a systemic reaction characterized by arterial hypotension, metabolic acidosis, decreased systemic vascular resistance, tachypnea and organ dysfunction. Sepsis can result from septicemia (i.e., organisms, their metabolic end-products or toxins in the blood stream), including bacteremia (i.e., bacteria in the blood), as well as toxemia (i.e., toxins in the blood), including endotoxemia (i.e., endotoxin in the blood). The term “bacteremia” includes occult bacteremia observed in young febrile children with no apparent foci of infection. The term “sepsis” also encompasses fungemia (i.e., fungi in the blood), viremia (i.e., viruses or virus particles in the blood), and parasitemia (i.e., helminthic or protozoan parasites in the blood). Thus, septicemia and septic shock (acute circulatory failure resulting from septicemia often associated with multiple organ failure and a high mortality rate) may be caused by a number of organisms.

The systemic invasion of microorganisms presents two distinct problems. First, the growth of the microorganisms can directly damage tissues, organs, and vascular function. Second, toxic components of the microorganisms can lead to rapid systemic inflammatory responses that can quickly damage vital organs and lead to circulatory collapse (i.e., septic shock) and oftentimes, death.

There are three major types of sepsis characterized by the type of infecting organism. Gram-negative sepsis is the most common and has a case fatality rate of about 35%. The majority of these infections are caused by Escherichia coli, Klebsiella pneumoniae and Pseudomonas aeruginosa. Gram-positive pathogens such as the Staphylococci and Streptococci are the second major cause of sepsis. The third major group includes fungi, with fungal infections causing a relatively small percentage of sepsis cases, but with a high mortality rate.

Many of these infections are acquired in a hospital setting and can result from certain types of surgery (e.g., abdominal procedures), immune suppression due to cancer or transplantation therapy, immune deficiency diseases, and exposure through intravenous catheters. Sepsis is also commonly caused by trauma, difficult newborn deliveries, and intestinal torsion (especially in dogs and horses).

Many patients with septicemia or suspected septicemia exhibit a rapid decline over a 24-48 hour period. Thus, rapid methods of diagnosis and treatment delivery are essential for effective patient care. Unfortunately, a confirmed diagnosis as to the type of infection traditionally requires microbiological analysis involving inoculation of blood cultures, incubation for 18-24 hours, plating the causative organism on solid media, another incubation period, and final identification 1-2 days later. Therefore, therapy must be initiated without any knowledge of the type and species of the pathogen, and with no means of knowing the extent of the infection.

It is widely believed that anti-endotoxin antibody treatment administered after sepsis is established may yield little benefit because these antibodies cannot reverse the inflammatory cascade initiated by endotoxin. In addition, the high cost of each antibody could limit physicians' use of a product where no clear benefit has been demonstrated. [K. A. Schulman et al., JAMA 266:3466-3471 (1991).] Furthermore, these endotoxin antibodies only target gram-negative sepsis, and no equivalent antibodies exist for the array of gram-positive organisms and fungi.

Clearly, there is a great need for agents capable of diagnosing and preventing and treating sepsis. It would be desirable if such agents could be administered in a cost-effective fashion. Furthermore, approaches are needed to combat all forms of sepsis.

### SUMMARY

The present invention relates to chimeric anti-CD14 antibodies and methods of using the same. In some embodiments, the present invention relates to the use of chimieric anti-CD14 antibodies in research, diagnostic, and therapeutic applications.

Embodiments of the present invention provides an isolated chimeric mouse human monoclonal antibody that binds to CD14, wherein said antibody has a variable light chain amino acid sequence selected from SEQ ID NO: 1 and sequences that are least 80% identical to SEQ ID NO:1 (e.g., at least 85%, 905, 91%, 92%, 93%, 94%, 95%, 96%, 97%, 98%, or 99% identifical to SEQ ID NO:1) and a variable heavy chain amino acid sequence selected from SEQ ID NO: 2 and sequences that are least 80% identical to SEQ ID NO:2 (e.g., at least 85%, 905, 91%, 92%, 93%, 94%, 95%, 96%, 97%, 98%, or 99% identifical to SEQ ID NO:2). In some embodiments, the present invention provides an isolated chimeric mouse human monoclonal antibody that binds to CD14, wherein said antibody has a variable light chain amino acid sequence selected from SEQ ID NO: 3 and sequences that are least 80% identical to SEQ ID NO:3 (e.g., at least 85%, 905, 91%, 92%, 93%, 94%, 95%, 96%, 97%, 98%, or 99% identifical to SEQ ID NO:3) and a variable heavy chain amino acid sequence selected from SEQ ID NO: 4 and sequences that are least 80% identical to SEQ ID NO:4 (e.g., at least 85%, 905, 91%, 92%, 93%, 94%, 95%, 96%, 97%, 98%, or 99% identifical to SEQ ID NO:4). In some embodiments, the antibody comprises a human IgG2/IgG4 hybrid C region. In some embodiments, the antibody has a constant light chain amino acid sequence selected from SEQ ID NO: 5 and sequences that are least 80% identical to SEQ ID NO:5 (e.g., at least 85%, 905, 91%, 92%, 93%, 94%, 95%, 96%, 97%, 98%, or 99% identifical to SEQ ID NO:5) and a constant heavy chain amino acid sequence selected from SEQ ID NO: 6 and sequences that are least 80% identical to SEQ ID NO:6 (e.g., at least 85%, 905, 91%, 92%, 93%, 94%, 95%, 96%, 97%, 98%, or 99% identifical to SEQ ID NO:6). In some embodiments, the antibody is an antibody fragment (e.g., Fab, Fab′, Fab′-SH, F(ab′)₂, Fv, or scFv variants). In some embodiments, the antibody is a full length antibody. In some embodiments, the antibody comprises an antibody fragment fused to a non-antibody molecule. In some embodiments, the non-antibody molecule is a human serum albumin polypeptide (e.g., variant polypeptide).

In some embodiments, the antibody is a humanized antibody. In some embodiments, the antibody inhibits at least one biological activity of CD14. In some embodiments, the antibody does not induce Fc-mediated side effects.

Further embodiments provide a pharmaceutical composition comprising any of the afore described antibodies. In some embodiments, the pharmaceutical composition further comprises an inhibitor of a complement component (e.g., C5). In some embodiments, the complement inhibitor is eculizumab, OmCI, or those shown in Table 3.

Additional embodiments provide uses and method of treating or preventing sepsis: administering the pharmaceutical composition of any one of claims 8 to 11 to a subject diagnosed with or at risk of sespsis.

Additional embodiments are described herein.

### BRIEF DESCRIPTION OF THE FIGURES

FIG. 1 shows expression of recombinant IgG2/4 hybrid Abs. (A-C) The Ab concentration in cell culture supernatant (mg/ml; s) and the Ab production rate (pg/cell/day; d) during a fed-batch expression period of 12 d were determined with ELISA. Data are given as mean and SEM for rMil2 (A; n=12), r18D11 (B; n=5) and raNIP (C; n=2). (D) Five hundred nanograms of each purified Ab was subjected to either reducing or nonreducing SDS-PAGE and stained with Coomassie Blue. (E) The same samples (10 ng) were subjected to nonreducing SDS-PAGE and immunoblotting using an Ab specific for the human IgG2 hinge region.

FIG. 2 shows functional characterization of anti-porcine CD14 Ab rMil2. (A) Whole porcine blood was incubated with 150 mg/ml FITC-conjugated Mil2 (FITC-Mil2) and increasing concentrations of unlabeled rMil2 (s), control IgG2/4 (eculizumab) (N), the original clone Mil2 (d) or mIgG2b isotype control (n). (B and C) Release of the proinflammatory cytokines IL-1b (B) and TNF (C) from porcine whole blood induced with 1 3 105/ml E. coli strain LE392 in presence of increasing concentrations of rMil2 (s), Mil2 (d), control IgG2/4 (N), or mIgG2b (n). (D) Porcine whole blood was incubated with increasing concentrations of rMil2 or Mil2, or ctrl IgG2/4 or mIgG2b isotype controls (up to 71.4 mg/ml). (E) Blood slides from samples containing 71.4 mg/ml rMil2 or Mil2 were stained with nuclear stain and investigated by light microscopy. (F) Porcine whole blood was incubated with 50 mg/ml rMil2 (s), Mil2 (d), control IgG2/4 (N) or mIgG2b (n).

FIG. 3 shows the effect of rMil2 in combination with C5-inhibitor OmCI on the inflammatory response in porcine blood in vitro. Plasma was analyzed for cytokines (A) TNF. (B) IL-1b. (C) IL-8. (D) TF expression on granulocytes was measured by flow cytometry and expressed as median fluorescence intensity (MFI).

FIG. 4 shows functional characterization of anti-human D14 Ab r18D11. (A) Binding of increasing concentrations of r18D11 (O), raNIP (N), 18D11 F(ab)92 (▴) or a control F(ab)92 (n) to monocytes was determined by the ability of the Abs to displace 10 mg/ml of the original clone 18D11 mIgG1 from its CD14 binding site in human whole blood. (B-D) Release of the proinflammatory cytokines IL-1b (B), TNF (C), and IL-6 (D) from human whole blood was induced with 100 ng/ml ultrapure LPS from E. coli O111:B4 in the presence of increasing concentrations of r18D11 (O) or the original clone 18D11 (▴). (E) Monocyte oxidative burst was measured with flow cytometry after adding the different Ab preparations to human whole blood.

FIG. 5 shows in vitro binding of recombinant IgG2/4 hybrid Abs to complement and Fc-receptors. Increasing concentrations of rMil2 (s), r18D11 (0) or raNIP (N) were incubated with (A) immobilized human C1q, (B) the human Fcg receptors FcgRI, (C) FcgRIIa (allotype His131), (D) FcgRIIb, (E) FcgRIIIa (allotype Val158), (F) FcgRIIIb, and (G, H) human (hFcRn) or (I, J) porcine FcRn (pFcRn) at acidic (pH 6.0) and neutral pH (pH 7.4).

FIG. 6 shows in vivo application of anti-porcine CD14 Abs Mil2 and rMil2. Healthy newborn piglets (A-D) were infused i.v. with increasing amounts of the original clone Mil2 (n=1) or rMil2 (n=1) and observed for 50 min. (A) After initial small doses within the first 10 min, Mil2 or rMil2 were given every 5 min for an additional 35 min. (B) Saturation of endogenous CD14 binding sites as a function of rMil2 (s) or Mil2 (d) concentration was measured by flow cytometry. (C) The heart rate (HR) was recorded in real time throughout the experiments. (D) Blood platelet counts are given as a function of rMil2 (s) or Mil2 (d) concentration. (E-I). One piglet was injected with a bolus dose of rMil2 before i.v. infusion with bacteria and one received saline as control.

### DETAILED DESCRIPTION OF CERTAIN EMBODIMENTS OF THE INVENTION

### Definitions

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The term “antibody” is used herein in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody and that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

An “effective amount” of an agent, e.g., a pharmaceutical formulation, refers to an amount effective, at dosages and for periods of time necessary, to achieve the desired therapeutic or prophylactic result.

The term “epitope” refers to the particular site on an antigen molecule to which an antibody binds.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR,” as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987).) Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, Md. (1991).) With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See Almagro and Fransson, Front. Biosci. 13:1619-1633 (2008).) Unless otherwise indicated, HVR residues and other residues in the variable domain (e.g., FR residues) are numbered herein according to Kabat et al., supra.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “isolated antibody” is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated nucleic acid” refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

I. Antibody Compositions

In some embodiments, the invention provides isolated antibodies that bind to CD14. In some embodiments, the antibodies are chimeric mouse/human antibodies. In some embodiments, the antibodies are monoclonal antibodies. The antibodies have variable regions that are specific for pig or human CD14. The variable region light chains are described by SEQ ID NOs: 1 or 3 or sequences that are at least 80% homologous to SEQ ID NOs: 1 or 3 (e.g., at least 80%, 85%, 90%, 91%, 92%, 93%, 94%, 95%, 96%, 97%, 98%, 99%, or 100% sequence identity to SEQ ID NOs: 1 or 3). The variable region heavy chains are described by SEQ ID NOs: 2 and 4 or sequences that are at least 80% homologous to SEQ ID NOs: 2 or 4 (e.g., at least 80%, 85%, 90%, 91%, 92%, 93%, 94%, 95%, 96%, 97%, 98%, 99%, or 100% sequence identity to SEQ ID NOs: 2 or 4).

In some embodiments, an anti-CD14 antibody is humanized. In one embodiment, an anti-CD14 antibody comprises a human acceptor framework, e.g. a human immunoglobulin framework or a human consensus framework. In certain embodiments, the human acceptor framework is the human VL kappa IV consensus (VL_{KIV}) framework and/or the VH framework VH₁. In certain embodiments, the human acceptor framework is the human VL kappa IV consensus (VL_{KIV}) framework and/or the VH framework VH₁ comprising an R71S mutation and an A78V mutation in heavy chain framework region FR3. In some embodiments, the light chain constant region is described by SEQ ID NO:5 and sequences that are at least 80% homologous to SEQ ID NO:5 (e.g., at least 80%, 85%, 90%, 91%, 92%, 93%, 94%, 95%, 96%, 97%, 98%, 99%, or 100% sequence identity to SEQ ID NO: 5).

In some embodiments, an anti-CD14 antibody comprises a heavy chain framework FR3 sequence selected. In some embodiments, an anti-CD14 antibody comprises a heavy chain framework FR3 sequence. In some such embodiments, the heavy chain variable domain framework is a modified human VH₁ framework. In some embodiments, the heavy chain constant region is described by SEQ ID NO:6 and sequences that are at least 80% homologous to SEQ ID NO:6 (e.g., at least 80%, 85%, 90%, 91%, 92%, 93%, 94%, 95%, 96%, 97%, 98%, 99%, or 100% sequence identity to SEQ ID NO: 6).

In a further aspect, the invention provides an antibody that binds to the same epitope as an anti-CD14 antibody provided herein.

In a further aspect of the invention, an anti-CD14 antibody according to any of the above embodiments is a monoclonal antibody, including a chimeric, humanized or human antibody. In one embodiment, an anti-CD14 antibody is an antibody fragment, e.g., a Fv, Fab, Fab′, scFv, diabody, or F(ab′)₂ fragment. In another embodiment, the antibody is a substantially full length antibody, e.g., an IgG1 antibody or other antibody class or isotype as defined herein.

In certain embodiments, a VH or VL sequence described herein contains substitutions (e.g., conservative substitutions), insertions, or deletions relative to the reference sequence, but an anti-CD14 antibody comprising that sequence retains the ability to bind to CD14. In certain embodiments, a total of 1 to 10 amino acids have been substituted, inserted and/or deleted. In certain embodiments, a total of 1 to 5 amino acids have been substituted, inserted and/or deleted.

In a further aspect of the invention, an anti-CD14 antibody according to any of the above embodiments is a monoclonal antibody, including a human antibody. In one embodiment, an anti-CD14 antibody is an antibody fragment, e.g., a Fv, Fab, Fab′, scFv, diabody, or F(ab′)₂ fragment. In another embodiment, the antibody is a substantially full length antibody, e.g., an IgG2a antibody or other antibody class or isotype as defined herein.

Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which VRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the VR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HuMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

Library-Derived Antibodies

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

Multispecific Antibodies

In certain embodiments, an antibody provided herein is a multispecific antibody, e.g. a bispecific antibody. Multispecific antibodies are monoclonal antibodies that have binding specificities for at least two different sites. In certain embodiments, one of the binding specificities is for CD14 and the other is for any other antigen. In certain embodiments, one of the binding specificities is for CD14 and the other is for CD3. See, e.g., U.S. Pat. No. 5,821,337. In certain embodiments, bispecific antibodies may bind to two different epitopes of CD14. Bispecific antibodies may also be used to localize cytotoxic agents to cells which express CD14. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting FAb” or “DAF” comprising an antigen binding site that binds to CD14 as well as another, different antigen (see, US 2008/0069820, for example).

Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

Substitution, Insertion, and Deletion Variants

In certain embodiments, antibody variants having one or more amino acid substitutions are provided. Sites of interest for substitutional mutagenesis include the VRs and FRs. Conservative substitutions are shown in the Table below under the heading of “preferred substitutions.” More substantial changes are provided in the Table below under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; Met; | Ile |
| | Ala; Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; | Leu |
| | Norleucine | |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more residues are mutated and the variant antibodies displayed on phage and screened for a particular biological activity (e.g. binding affinity).

Alterations (e.g., substitutions) may be made e.g., to improve antibody affinity. Such alterations may be made in “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves directed approaches, in which several residues (e.g., 4-6 residues at a time) are randomized. Residues involved in antigen binding may be specifically identified, e.g., using alanine scanning mutagenesis or modeling.

In certain embodiments, substitutions, insertions, or deletions may occur within one or more VRs so long as such alterations do not substantially reduce the ability of the antibody to bind antigen. For example, conservative alterations (e.g., conservative substitutions as provided herein) that do not substantially reduce binding affinity may be made in VRs. Such alterations may be outside of VR “hotspots” or SDRs. In certain embodiments of the variant VH and VL sequences provided above, each VR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex is used to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express Fc(RIII only, whereas monocytes express Fc(RI, Fc(RII and Fc(RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. No. 5,648,260; U.S. Pat. No. 5,624,821; and WO 94/29351 concerning other examples of Fc region variants.

Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, or to create an immunoconjugate.

Antibody Derivatives

In certain embodiments, an antibody provided herein may be further modified to contain additional nonproteinaceous moieties or non-antibody proteins that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

In some embodiments, antibodies or antibody fragments (e.g., antigen binding fragments) are fused or conjugated to human serum albumin (See e.g., U.S. Pat. Nos. 7,785,599 and 7,550,432). Albumin binds in vivo to the neonatal Fc receptor (FcRn) and this interaction is known to be important for the plasma half-life of albumin (Chaudhury et al 2003; Montoyo et al., 2009). FcRn is a membrane bound protein, and has been found to salvage albumin as well as IgG from intracellular degradation (Roopenian D. C. and Akilesh, S. (2007), Nat. Rev. Immunol 7, 715-725.). Thus, FcRn is a bifunctional molecule that contributes to the maintaining the high level of IgG and albumin in serum of mammals such as humans.

Human serum albumin (HSA) has been well characterised as a polypeptide of 585 amino acids, the sequence of which can be found in Peters, T., Jr. (1996) All about Albumin: Biochemistry, Genetics and Medical, Applications, Academic Press, Inc., Orlando. It has a characteristic binding to its receptor FcRn, where it binds at pH 6.0 but not at pH 7.4. The serum half-life of HSA has been found to be approximately 19 days. A natural variant having lower plasma half-life has been identified (Biochim Biophys Acta. 1991, 1097:49-54) having the substitution D494N. This substitution generated an N-glycosylation site in this variant, which is not present in the wild type HSA.

Albumin has a long serum half-life and because of this property it has been used for drug delivery. Albumin has been conjugated to pharmaceutically beneficial compounds (WO0069902A), and it was found that conjugate had maintained the long plasma half-life of albumin so the resulting plasma half-life of the conjugate has generally been found to be considerably longer than the plasma half-life of the beneficial therapeutic compound alone.

Further, albumin has been fused to therapeutically beneficial peptides (WO 01/79271 A and WO 03/59934 A) with the typical result that the fusion has the activity of the therapeutically beneficial peptide and a long plasma half-life considerably longer than the plasma half-life of the therapeutically beneficial peptides alone.

Recombinant Methods and Compositions

Antibodies may be produced using recombinant methods and compositions, e.g., as described in U.S. Pat. No. 4,816,567. In one embodiment, isolated nucleic acid encoding an anti-CD14 antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an anti-CD14 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-CD14 antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

Assays

Anti-CD14 antibodies provided herein may be identified, screened for, or characterized for their physical/chemical properties and/or biological activities by various assays known in the art. In some embodiments, the experiments described in Example 1 are utilized to screen antibodies for activity.

In one aspect, an antibody of the invention is tested for its antigen binding activity, e.g., by known methods such as ELISA, BIACore®, FACS, or Western blot.

In another aspect, competition assays may be used to identify an antibody that competes with any of the antibodies described herein for binding to CD14. In certain embodiments, such a competing antibody binds to the same epitope (e.g., a linear or a conformational epitope) that is bound by an antibody described herein. Detailed exemplary methods for mapping an epitope to which an antibody binds are provided in Morris (1996) “Epitope Mapping Protocols,” in Methods in Molecular Biology vol. 66 (Humana Press, Totowa, N.J.).

In an exemplary competition assay, immobilized CD14 is incubated in a solution comprising a first labeled antibody that binds to CD14 (e.g., any of the antibodies described herein) and a second unlabeled antibody that is being tested for its ability to compete with the first antibody for binding to CD14. The second antibody may be present in a hybridoma supernatant. As a control, immobilized CD14 is incubated in a solution comprising the first labeled antibody but not the second unlabeled antibody. After incubation under conditions permissive for binding of the first antibody to CD14, excess unbound antibody is removed, and the amount of label associated with immobilized CD14 is measured. If the amount of label associated with immobilized CD14 is substantially reduced in the test sample relative to the control sample, then that indicates that the second antibody is competing with the first antibody for binding to CD14. See Harlow and Lane (1988) Antibodies: A Laboratory Manual ch. 14 (Cold Spring Harbor Laboratory, Cold Spring Harbor, N.Y.).

Pharmaceutical Formulations

Pharmaceutical formulations of an anti-CD14 antibody as described herein are prepared by mixing such antibody having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredient as necessary for the particular indication being treated, preferably those with complementary activities that do not adversely affect each other. For example, in some instances, it may be desirable to further provide an inhibitor of a complement component (e.g., OMCI or those described in Table 3). In some embodiments, complement inhbitors are formulated in the same or different pharmeutical compositions (e.g., for co-administration).

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

II. Therapeutic and Research Methods and Compositions

Embodiments of the present disclosure provide methods and uses for treating or preventing sepsis (e.g., using the monoclonal antibodies described herein). In some embodiments, the subject has been diagnosed with sepsis. In some embodiments, the subject is suspected of having sepsis. In some embodiments, the subject is at risk of sepsis and the treatment prevents sepsis.

In some embodiments, sepsis is treated using a combination of one of the monoclonal antibodies described herein and an inhibitor of complement (e.g, C5). Examples include, but are not limited to, OMCI and the inhibitors in Table 3.

In another aspect, an anti-CD14 antibody for use as a medicament is provided. In a further aspect, the invention provides for the use of an anti-CD14 antibody in the manufacture or preparation of a medicament. In one embodiment, the medicament is for treatment of sepsis.

An “individual” according to any of the above embodiments may be a human.

Such combination therapies noted above encompass combined administration (where two or more therapeutic agents are included in the same or separate formulations), and separate administration, in which case, administration of the antibody of the invention can occur prior to, simultaneously, and/or following, administration of the additional therapeutic agent and/or adjuvant.

An antibody of the invention (and any additional therapeutic agent) can be administered by any suitable means, including parenteral, intrapulmonary, and intranasal. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of sepsis, the appropriate dosage of an antibody of the invention (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 15 mg/kg (e.g. 0.1 mg/kg-10 mg/kg) of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody or would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose, followed by one or more lower doses may be administered. However, other dosage regimens may be useful. The progress of this therapy is easily monitored by conventional techniques and assays.

Embodiments of the present disclosure further provide research uses (e.g., to study sepsis or other CD14 mediated disorders) in animal (porcine) or in vitro.

Articles of Manufacture

In another aspect of the invention, an article of manufacture containing materials useful for the treatment, prevention and/or diagnosis of sepsis is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the disorder and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution or dextrose solution. It may further include other materials desirable from a commercial and user standpoint, including other buffers, diluents, filters, needles, and syringes.

### EXAMPLES

### Example 1

### Materials and Methods

Abs and Inhibitors

Commercial anti-CD14 Abs and isotype controls were purchased at Diatec Monoclonals AS (Oslo, Norway) and AbD Serotec (Kidlington, U.K.) as follows: mouse anti-human CD14 IgG1 clone 18D11 (Diatec), 18D11 IgG1 F(ab)92 (Diatec), mouse anti-porcine CD14 IgG2b clone Mil2 (Serotec) and FITC-conjugated Mil2 (Serotec), isotype controls mouse IgG1 (Diatec), mouse IgG1 F(ab)92 (Diatec), mouse IgG2b (Diatec), and FITC conjugated mouse IgG2b (Serotec). the fully humanized anti-05 IgG2/4 Ab eculizumab (Soliris), purchased from Alexion Pharmaceuticals (Cheshire, Conn.), was used as isotype control for recombinant IgG2/4 and humanized anti-CD20 IgG1 Ab rituximab (MabThera) from Roche (Welwyn Garden City, U.K.) as control for ELISA-based binding studies.

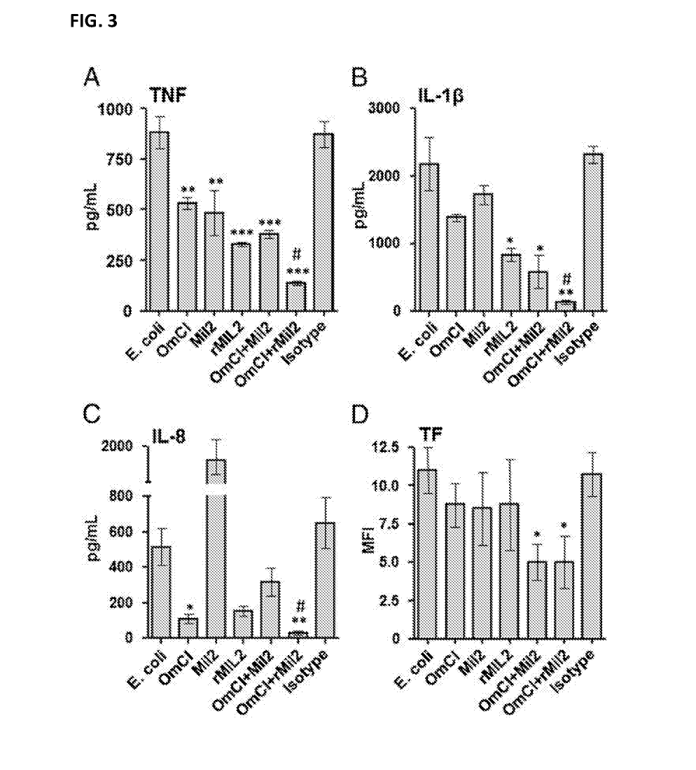

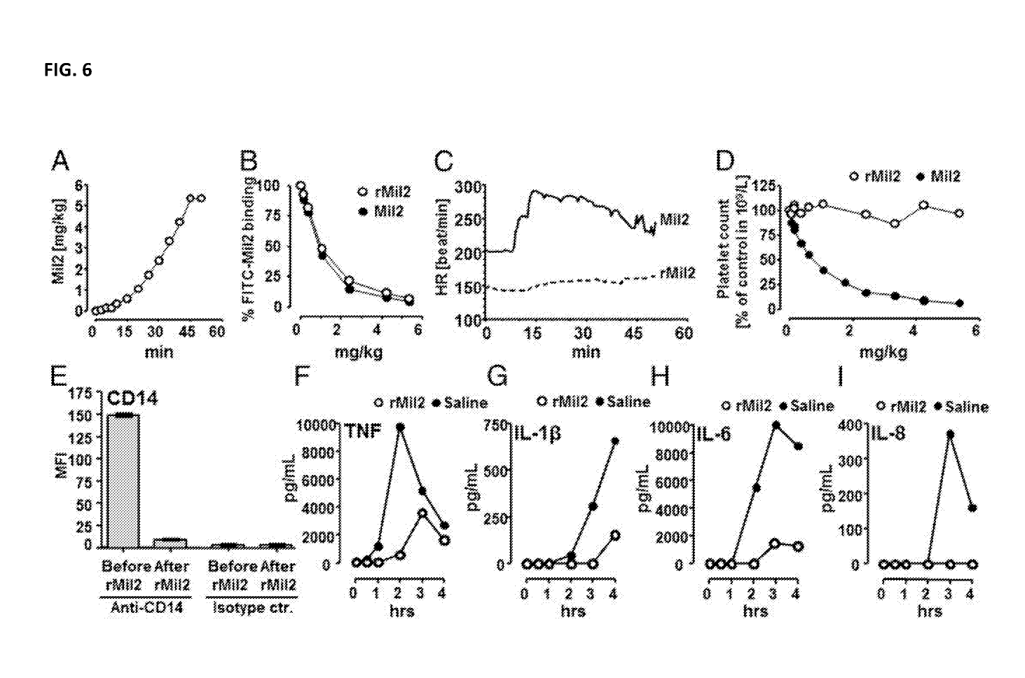

The rMil2 Ab preparation used in the experiments shown in FIGS. 3 and 6 was produced by ExcellGene SA (Monthey, Switzerland). This Ab consists of the same amino acid sequence presented in Table 4, but was expressed in a CHO cell-expression system. It was pure and free of single chains, as confirmed by SDS-PAGE (not shown).

Goat-anti human IgG k pooled antisera, HRP-conjugated goat-anti human IgG Fc pooled antisera, HRP-conjugated goat anti-mouse IgG Ab, and PE-conjugated anti-mouse IgG were purchased from Southern Biotech (Birmingham, Ala.). Mouse monoclonal anti-human IgG2 Ab (clone 3C7) was purchased from Hytest (Turku, Finland). Endotoxin-free recombinant bacterial OmCI (also known as coversin) (Nunn, et al., 2005. J. Immunol. 174: 2084-2091), a 16.8-kDa protein, was provided by Varleigh Immuno Pharmaceuticals (Jersey, Channel Islands). OmCI has been shown to inhibit complement activation effectively in pigs (Barratt-Due, et al., 2011 J. Immunol. 187: 4913-4919).

Variable gene retrieval and cloning of recombinant anti-CD14

Abs

Original hybridoma cell clones were generated in the laboratories of the coauthors T. E. (18D11) and C. R. S. (Mil2). After brief culture, the cells were harvested and total RNA was extracted using mirVana (Life Technologies, Ambion, Austin, Tex.). Variable genes were specifically reverse transcribed from 500 ng total RNA using SuperScript II reverse transcriptase and oligonucleotide primers, which were designed to bind downstream of the variable genes in conserved sequences encoding the constant regions of heavy and light chains. After removal of input RNA from the sample by RNaseH digestion (New England Biolabs, Hedfordshire, U.K.), poly dCTP 39-tailing of the cDNA was performed using rTerminal transferase (Roche Diagnostics, Mannheim, Germany), and fragments containing the variable gene segments were amplified by nested PCR using Phusion DNA polymerase (Finnzymes, Vantaa, Finland) and new sets of primers containing BglII and MluI restriction sites. The amplicons were inserted in cloning vectors before sequencing analyses. All primers were synthesized by Sigma-Aldrich (Steinheim, Germany; Table 5).

A well-established protocol (Norderhaug, et al., 1997. J. Immunol. Methods 204: 77-87) was used to subclone the murine variable heavy and variable L chain genes into pLNOH2 and pLNOK expression vectors, respectively. The sequence encoding the human IgG2/4 hybrid constant H chain was consistent with the literature (Mueller, et al., 1997. Mol. Immunol. 34: 441-452) and inserted into pLNOH2. All genes were synthesized by GenScript (Piscataway, N.J.). The control Ab specific for a hapten (4-hydroxy-3-iodo-5-nitrophenylacetic acid [NIP]) was also expressed from pLNOH2 and pLNOK (Norderhaug, et al., 1997. J. Immunol. Methods 204: 77-87). Thus, two plasmids were generated for the expression of each of the three recombinant Abs, which target human CD14 (recombinant 18D11, r18D11), porcine CD14 (recombinant Mil2, rMil2), or NIP (recombinant anti-NIP, raNIP). For transfection, plasmid DNA was purified using EndoFree Plasmid Maxi or Mega Kit from Qiagen (Hilden, Germany). Amino acid sequences of the recombinant Abs and related IMGT accession numbers are displayed in 1 Table 4.

Cell Culture

Adherent HEK293-EBNA cells were subcultured at 5% CO2 and 37° C. using DMEM containing 4.5 g/L L-glucose (Lonza, Verviers, Belgium) and substituted with 10% FBS FBS Gold (PAA Laboratories, Pasching, Austria), 4 mM L-glutamine (Lonza), 10,000 Um′ penicillin, and 10,000 mg/ml streptomycin (Lonza). The day before transfection, 4 3 106 cells were seeded in a 75-cm2 tissue culture flask (Techno Plastic Products, Trasadingen, Switzerland) and grown for an additional 24 h to reach 90% confluency. Cotransfection with light and H chain encoding plasmid DNA in an equimolar ratio was performed in serum-free OptiMEM (Life Technologies, Paisley, U.K.) using Lipofectamine 2000 (Life Technologies, Invitrogen Carlsbad, Calif.) following the manufactures instructions. Transient expression was performed in a fed-batch procedure with harvest of cell culture supernatant every third day over 12 d. The cells were detached and centrifuged for 5 min at 230 3 g followed by careful aspiration of the supernatant and immediate resuspension and reseeding of the cells in 12 ml fresh OptiMEM. The supernatant was stored at 220° C. until Ab purification. Cell viability and count was monitored throughout subculture and before every harvest using Countess Automated Cell Counter (Life Technologies, Invitrogen).

IgG Purification

Concentrators, spin columns, and kits for purification of recombinant Abs and subsequent buffer exchange were purchased from Thermo Scientific, Pierce (Pierce Biotechnology, Rockford, Ill.). The combined supernatants of each expression culture were centrifuged for 10 min at 1500 3 g and subsequently filtrated using a sterile vacuum filter system with a 0.22-mm cellulose acetate membrane (Corning Glass Works, Corning, N.Y.). Then, solutions were concentrated using Pierce's concentrators with a 20-kDa m.w. cutoff (MWCO), and OptiMEM was exchanged to sterile PBS using 10 ml Zeba Desalt Spin Columns with a 7-kDa MWCO. The recombinant Abs were purified using an NAb Protein A Plus Spin Kit with a binding capacity of 7 mg IgG per 0.2 ml resin. Ab-containing fractions were combined before buffer exchange to sterile PBS using 2 ml Zeba Desalt Spin Columns with a 7-kDa MWCO and optional upconcentration to 0.5-1 mg/ml using Amicon Ultra 0.5-ml spin columns with a 50-kDa MWCO (Millipore, Carrigtwohill, Ireland). Endotoxin levels in the final batch preparations were less than 0.04 EU/ml, determined using QCL-1000 (Lonza, Walkersville, Md.). Ab expression was monitored using the k-chain—specific goat-anti human IgG pooled antisera diluted to 1 mg/ml in carbonate buffer as capture and the HRP-conjugated, Fc-specific goat-anti human IgG pooled antisera diluted 1:8000 in PBS for detection (see above).

SDS-PAGE and Western Blot

All materials were purchased from Bio-Rad Laboratories AB (Hercules, Calif.), except where indicated differently. Purified Ab fractions were separated using SDS-PAGE on a Mini-PROTEAN Tetra Cell using Mini-PROTEAN Precast Gels (4-15%) and Tris Glycin SDS buffer. Samples were diluted in 2× Laemmli buffer with or without 5% b-mercaptoethanol. Gels were stained with Biosafe Coomassie G250 following the manufacturer's instructions. Alternatively, proteins were blotted onto a Hybond ECL nitrocellulose membrane (Amersham Pharmacia Biotech, Buckinghamshire, U.K.). After blocking with 5% nonfat dry milk, the membrane was incubated with primary mouse anti-human IgG2 Ab (clone 3C7; 1 mg/ml) and secondary HRP-conjugated goat anti-mouse IgG Ab. Detection of specific bands by ECL was performed using SuperSignal West Dura (Pierce Biotechnology). Images were taken with a ChemiDoc XRS+system.

CD14 Binding in Flow Cytometry

Fresh human or porcine whole blood was drawn into tubes containing the anticoagulants lepirudin (Refludan; Pharmion, Copenhagen, Denmark) or EDTA, respectively. Blood samples were preincubated for 10 min with Ab, #20 mg/ml anti-human CD14 Abs r18D11, 18D11 F(ab)92 (batch 2068, lot 1383), or isotype controls raNIP and mIgG1 F(ab)92 (lot 1501), or 100 mg/ml anti-porcine CD14 Abs rMil2, Mil2 (lot 1106), or the isotype controls 4770 RECOMBINANT ANTI-CD14 Abs Downloaded from University of Oslo, Library of Medicine and Health Science on Jan. 24, 2014 for IgG2/4 and mIgG2b, eculizumab (Soliris) and mIgG2b (lot 1631), respectively. Endotoxin-free Dulbecco phosphate buffered saline, PBS (Sigma-Aldrich), was used for diluting the Abs. Subsequently, blood samples were incubated for another 15 min in the presence of detection Ab, 10 mg/ml 18D11 (batch 719, lot 3110) or 150 mg/ml FITC-conjugated Mil2 (batch 1107). Their binding was detected either by using a secondary PEconjugated anti-mouse IgG or through direct FITC fluorescence. Erythrocytes were lysed using FACS Lysing solution (BD Biosciences, Franklin Lakes, N.J.; human blood) or a solution of 0.16 M ammonium chloride, 10 mM sodium bicarbonate, 0.12 mM EDTA (Tritiplex III) and 0.04% (v/v) paraformaldehyde (porcine blood). In subsequent flow cytometry analyses, human monocyte and porcine granulocyte populations were selected based on the forward scatter-side scatter dot plot, and CD14 binding was recorded as mean or median fluorescence intensity, respectively. In contrast to humans, porcine CD14 is constitutively expressed on mature granulocytes (32, 33). Fluorescence intensities in the presence of the fluorescently labeled Abs only were set to 100%. Flow cytometry analyses on human samples were performed using an LSRII and FACSDiva software version 5.0.3; porcine samples were analyzed on a FACSCalibur using Cell Quest Pro version 5.2.1 for data acquisition (all from BD Biosciences).

Construction, Production, and Purification of Recombinant Soluble FcRn and FcgR Variants

The vector containing a truncated version of human FcRn (hFcRn) H chain cDNA encoding the three ectodomains (a1-a3) genetically fused to a cDNA encoding the Schistosoma japonicum GST has been described earlier (Berntzen, et al., 2005. J. Immunol. Methods 298: 93-104). The vector-denoted pcDNA3-hFcRn-GST-hb2m-origin of replication (oriP) also contains a cDNA encoding human b2-microglobulin and the EBV oriP. A truncated cDNA segment encoding the extracellular domains of porcine FcRn (pFcRn) was synthesized by Genscript and subcloned into the pcDNA3-GST-hb2m-oriP vector using the restriction sites EcoRI and XhoI. Vectors encoding the ectodomains of human FcgRI, FcgRIIa, FcgRIIb, FcgRIIIa, and FcgRIIIb fused to GST have been described previously (Berntzen, et al., 2005. J. Immunol. Methods 298: 93-104; Andersen, et al., 2012. J. Biol. Chem. 287: 22927-22937).

All recombinant soluble receptors were produced by transient transfection of HEK293-EBNA cells, and secreted receptors were purified using a GSTrap column as described previously (Berntzen, et al., 2005. J. Immunol. Methods 298: 93-104).

ELISA for C1q, FcgR, and FcRn Binding

Ninety-six-well plates (Nunc, Roskilde, Denmark) were coated with serial dilution of the Abs (6.0-0.09 mg/ml) and incubated overnight at 4° C. followed by washing three times with PBS/Tween (pH 7.4). The wells were blocked with 4% skimmed milk (Neogen Europe, Auchincruive, U.K.) for 1 h at room temperature and then washed in PBS/Tween (pH 6.0). Purified hFcRn-GST or pFcRn-GST (1 mg/ml) were diluted in 4% skimmed milk in PBS/Tween (pH 6.0) and preincubated with an HRP-conjugated anti-GST Ab (GE Healthcare U.K., Buckinghamshire, U.K.) diluted 1:5000 and added to the wells. The plates were incubated for 1 h at room temperature and washed with PBS/Tween (pH 6.0). Bound receptor was detected by adding 100 ml of 3,39,5,59-tetramethylbenzidine substrate (Calbiochem-Novabiochem, Nottingham, U.K.). The absorbance was measured at 450 nm using a Sunrise TECAN spectrophotometer. The assay described above was also performed using PBS/Tween (pH 7.4) in all steps. The same setup was used with GST-fused versions of hFcgRI, hFcgRIIa (allotype His131), hFcgRIIb, hFcgRIIIa (allotype Val158), and hFcgRIIIb (1 mg/ml each). In addition, a biotinylated human C1q (hC1 q; 4 mg/ml) preparation was incubated with the Abs and detected using ALP-conjugated streptavidin (GE Healthcare). Absorbance was measured at 405 nm.

Whole Blood Ex Vivo Model of Inflammation

The whole blood model has been described in detail previously (36). Fresh human venous or porcine arterial blood was drawn directly into tubes containing the anticoagulant lepirudin (Pharmion) at a final concentration of 50 mg/ml. In 1.8-ml Cryo Tube vials (Nunc, Roskilde, Denmark), the blood was preincubated with up to 20 mg/ml anti-human CD14 (r18D11 or 18D11), or 50 mg/ml anti-porcine CD14 (rMil2 or Mil2) at 37° C. for 10 min prior to an additional 2-h incubation in the presence of 100 ng/ml ultrapure LPS from Escherichia coli 0111:B4 (InvivoGen, San Diego, Calif.) for human blood or 1 3 105/ml heat-inactivated E. coli (strain LE392, ATCC33572) for porcine blood. As negative controls, PBS with MgCl2 and CaCl2 (Sigma-Aldrich) and isotype controls were used. Adverse effects were tested using nonactivated whole blood samples. After the addition of 10 mM (human) or 20 mM (porcine) EDTA, plasma was gained by 15 min centrifugation at 3220 3 g and 4° C. Levels of TNF, IL-6, and IL-1b in human plasma were determined using Bioplex technology (Bio-Rad Laboratories AB). Levels of TNF, IL-1b, and IL-8 in porcine plasma were determined using ELISA (Quantikine, R&D Systems, Minneapolis, Minn.). Platelet count was quantified by impedance using a CELL-DYN Sapphire hematology analyzer (Abbott Laboratories, Abbott Park, Ill.).

Effect of rMil2 combined with the complement C5 inhibitor OmCI on cytokine production in porcine whole blood ex vivo

Whole blood was incubated with 1 3 106 E. coli per milliliter for 2 h at 37° C. in the absence or presence of inhibitors and controls. TNF, IL-1b, and IL-8 blood was analyzed as described above.

Effect of rMil2 combined with the complement C5 inhibitor OmCI on leukocyte tissue factor expression

For analysis of leukocyte expression of tissue factor (TF), porcine whole blood was incubated with 5 3 106 E. coli per milliliter in the absence or presence of inhibitors and controls. After incubation, the tubes were put on ice, citrate was added to stop the activation, and the samples further analyzed by flow cytometry. One sample was split into two tubes and stained with sheep anti-human TF (Affinity Biologicals, Ancaster, Canada) and control sheep IgG (Sigma-Aldrich, Saint Louis, Mo.), respectively. All samples were incubated for 30 min at 4° C., and red cells were lysed and centrifuged at 300 3 g for 5 min at 4° C. The cells were washed with PBS (0.1% BSA; BioTest, Dreieich, Germany). Samples were further stained with rabbit anti-sheep IgG-PE conjugate (Santa Cruz Biotechnology, Dallas, Tex.) for an additional 30 min at 4° C. and then washed twice as described above. The cells were resuspended in PBS (0.1% BSA) before they were run at the flow cytometer (FACSCalibur; Becton Dickinson, Franklin Lakes, N.J.). Granulocytes were gated in a forward scatter-side scatter dot plot, and TF expression was given as median fluorescence intensity.

In vivo application of anti-porcine CD14 Abs Norwegian domestic piglets (Sus scrofa domesticus, outbred stock) with a weight of 2.2 kg were isolated at the day of intervention. Anesthesia was induced with 5% sevoflurane in a mixture of air and oxygen until sleep. After establishment of an i.v. line, the piglets received fentanyl (15-20 mg/kg) were tracheotomized in the supine position, and a microcuffed endotracheal tube from Kimberly-Clark (Roswell, GA) with inner diameter of 4 mm was inserted. Maintenance anesthesia was provided with an infusion of fentanyl (50 mg/kg/h, and isoflurane 1-2% in oxygen-enriched air administered from a Leon plus ventilator from Heinen and Loevenstein (Bad Ems, Germany). An artery line was inserted in the right or left carotid artery for blood sampling during the experiments and for continuous measurement of mean artery pressure. The piglets were monitored with electrocardiography and pulse oximetry. Ventilator settings were adjusted to maintain 7.40 pH and oxygen saturation above 96%. Hemodynamic parameters were collected using ICUpilot software, CMA Microdialysis (Stockholm, Sweden) every 30 s. To compensate for hydration needs, the animals received a background infusion of isotonic sodium glucose solution, Salidex (Braun Medical A/S, Vestskogen, Norway) at 10 ml/kg/h. To compare the effect of the Mil2 and the rMil2 on healthy piglets, increasing amounts of a stock solution of 1 mg/ml rMil2 or Mil2 (batch 1106) were injected i.v. into two piglets at indicated times to a maximum dose of 5.36 mg/kg, and arterial blood samples were collected, in tubes containing the anticoagulants EDTA or citrate. To investigate the biological effect of rMIL2 on the inflammatory response, two piglets underwent the E. coli sepsis regimen as described previously (17). One control piglet was gives saline, and one piglet was given a bolus dose of 5 mg/kg rMil2 before infusion of the bacteria.

Data Presentation and Statistics

All graphs were generated and statistical analyses were performed using GraphPad Prism version 5.03 from GraphPad Software (San Diego, Calif.). If not indicated differently, arithmetic mean values and SEM are displayed. Statistical significance was calculated usingANOVA and Tukey, Dunnett, or Bonferroni posttest analysis for subgroup comparison as indicated in the figure legends. Student t test was used to compare combined inhibition of anti-CD14 and OmCI compared with the two single inhibitions.

Results

Cloning and Expression of Recombinant Anti-CD14 Abs

Recombinant anti-porcine CD14 (rMil2) and anti-human CD14 (r18D11) Abs were generated, as both mouse human chimeras with murine variable and human constant regions (Table 4). For both region, the H chain C region (CH) was chosen such that the CH1 and hinge regions were from IgG2, whereas the CH2 and CH3 domains were from IgG4. The variable genes encoding the Ab specificities are identical to those of the originalmurine clones 18D11 and Mil2 (Table 4). raNIP with the same C region was also generated and included as isotype control in further studies.

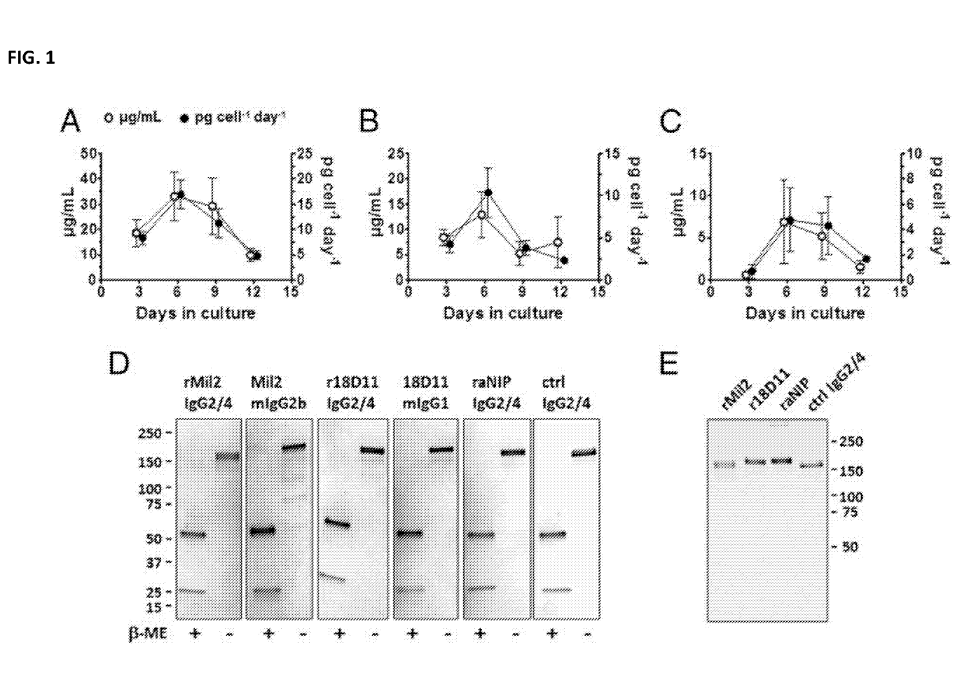

All Abs were readily expressed in adherent HEK293-EBNA cells after transient transfection, although at different levels. During expression under serum-free conditions, ˜20 mg/ml rMil2 was produced, while r18D11 and raNIP were produced at 4-8 mg/ml (FIG. 1A-C). For rMil2, production reached a maximum of 15 pg/cell/d between days 6 and 9 (FIG. 1A). The recombinant Abs were purified from the cell culture supernatant, subjected to re-ducing and nonreducing SDS-PAGE, and compared with commercially available batches of their original murine clones (FIG. 1D). The recombinant Abs were also detected by an anti-human IgG2 hinge Ab after Western blotting (FIG. 1E).

Functional Characterization of the Recombinant Anti-Porcine CD14 Ab rMil2

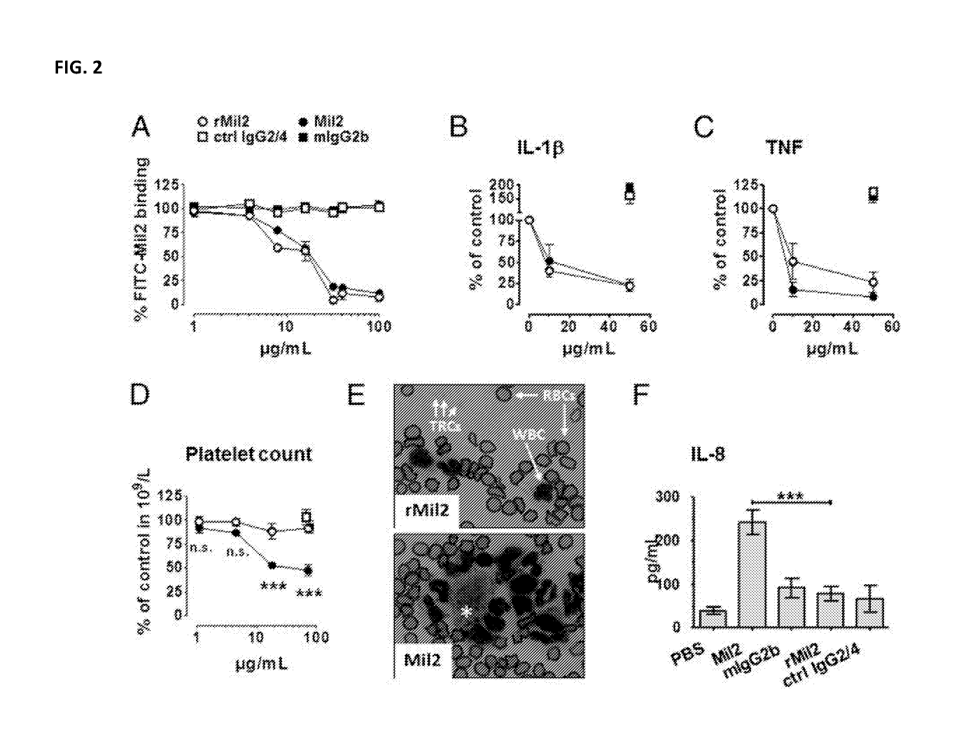

Whole blood from healthy pigs was used to study Ag-binding and CD14-blocking effects of the recombinant anti-porcine CD14 Ab rMil2. rMil2 effectively bound to and displaced the original clone, Mil2, from CD14+ granulocytes (FIG. 2A). Both rMil2 and Mil2 competed equally well with FITC-conjugated Mil2 in binding to CD14, and they blocked nearly 50% of the binding sites at 15 mg/ml. At this concentration, direct binding of rMil2 to porcine granulocytes was saturated (not shown). Furthermore, rMil2 effectively inhibited the proinflammatory cytokine response in whole blood induced by 1 3 105 cells/ml heat-inactivated E. coli (FIG. 2B, 2C). Therefore, it was as effective as Mil2 in the block of IL-1b release, and slightly less inhibitory on TNF release. In the presence of 10 mg/ml and 50 mg/ml of either Ab, IL-1b and TNF plasma levels were reduced by at least 75% and 50%, respectively.

Next, unwanted IgG-Fc mediated effects of rMil2 and Mil2 in the absence of inflammatory stimuli (FIG. 2D-F) were assayed. A dose-dependent drop in platelet counts for Mil2 (FIG. 2D) was observed. This highly significant drop was the result of platelet activation and aggregation, and platelet aggregates surrounded by leukocytes were observed in blood slides from the same samples (FIG. 2E). In addition, Mil2 induced a strong spontaneous IL-8 release (FIG. 2F). Neither IL-8 secretion nor platelet drop nor aggregation was observed in the presence of rMil2. None of the Abs induced significant complement activation, measured as terminal C5b-9 complement complex formation (not shown).

Effect of rMil2 in Combination with the Complement C5 Inhibitor OmCI on the Inflammatory Response

Based on recent promising data supporting a combined inhibition of CD14 and the complement system as a therapeutic approach for inflammatory conditions (Barratt-Due, et al., 2013. J. Immunol. 191: 819-827), the effect of the original Mil2 and the rMil2 werealone and in combination with the complement C5 inhibitor OmCI. Porcine whole blood was incubated with E. coli and the cytokine response (TNF, IL-1b, IL-8) and expression of granulocyte TF were studied (FIG. 3). TNF release was significantly reduced by a single treatment with OmCI and original Mil2 (p, 0.01), with rMil2, and with OmCI combined with either of the Mil2 Abs (p, 0.001; FIG. 3A). The combination of OmCI and rMil2 was the most effective inhibitory regimen (81% inhibition as compared with E. coli; p, 0.001) and significantly more effective than OmCI or rMIL2 alone (p=0.001 and p=0.004, respectively). IL-1b release was not significantly reduced by a single treatment with OmCI or original Mil2, but rMil2 alone and OmCI combined with either Mil2 Abs significantly reduced the production (p, 0.05; FIG. 3B). The combination of OmCI and rMil2 was the most effective inhibitory regimen (94% inhibition as compared with E. coli; p, 0.01) and significantly more effective than OmCI or rMil2 alone (p=0.002 and p=0.019, respectively). IL-8 release was significantly reduced to almost baseline by OmCI alone (p, 0.05), whereas original Mil2 markedly enhanced the release (FIG. 3C), consistent with the adverse effects of IL-8 by the original Ab observed previously (FIG. 2F). The inhibition seen with rMil2 alone seemed to be substantial, but did not reach statistical significance, presumably because of type II error. Notably, the combination of OmCI and rMil2 was again the most effective inhibitory regimen (94% inhibition as compared with E. coli; p, 0.01) and significantly more effective than OmCI or rMil2 alone (p=0.033 and p=0.008, respectively). TF, as expressed by neutrophils, was significantly reduced only by the combined inhibition of OmCI and the two anti-CD14 Abs (FIG. 3D; p, 0.05), rMil2 being similarly effective as the original Mil2.

Functional Characterization of Recombinant Anti-Human CD14 r18D11

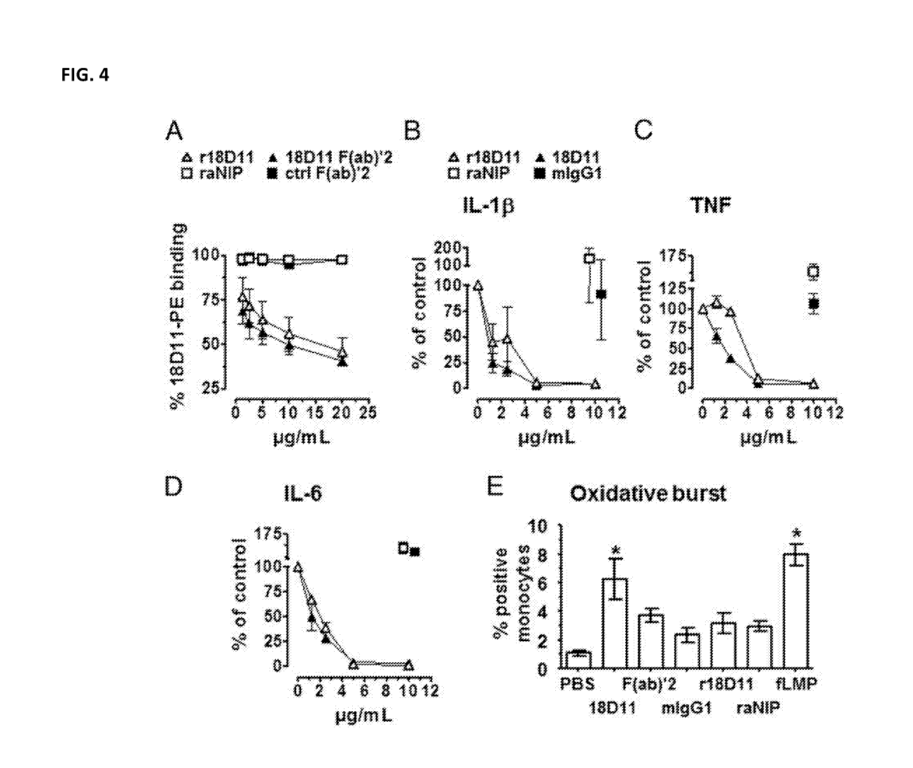

The recombinant anti-human CD14 Ab, r18D11, was tested with respect to Ag binding and inhibition of CD14-mediated cytokine release. It dose-dependently outcompeted the binding of the original clone, 18D11, to CD14-positive sites on human monocytes (FIG. 4A). The same was observed with a F(ab)92 fragment of the original clone. This indicates that the Abs bind to the same epitope, as expected. Equimolar amounts of r18D11 (10 mg/ml) displaced 50% of 18D11 from its binding sites. The lower competitive activity of 10 mg/ml r18D11 compared with 10 mg/ml of the F(ab)92 fragment of the murine clone is due to difference in molarities. Neither raNIP nor a control F(ab)92 fragment bound human CD14 (FIG. 4A). Furthermore, both 18D11 and r18D11 inhibited E. coli ultrapure LPS-induced release of the proinflammatory cytokines IL-1b, TNF, and IL-6 in human whole blood, in a dose-dependent manner (FIG. 4B-D). Maximum inhibitory effects were reached at an Ab concentration of 10 mg/ml, at which the recombinant clone was as effective as the original clone. Again, neither raNIP nor control mIgG1 inhibited LPS-induced cytokine release. Induction of unwanted effects, such as complement activation and oxidative burst, was tested using nonstimulated human whole blood supplemented with 10 mg/ml Ab. The original clone 18D11, r18D11, and raNIP induced the same low level of complement activation, whereas the F(ab)92 fragment of 18D11 and a mIgG1 isotype control did not (not shown). Monocyte oxidative burst, however, was significantly induced by the original 18D11 clone, comparable to that of the positive fMPL control (FIG. 4E). Notably, r18D11 did not induce significant oxidative burst, and was comparable with the F(ab9)2 18D11, the isotype IgG1 control and the IgG2/4 chimeric negative control raNIP (FIG. 4E).

Binding of rMil2 and r18D11 to Complement Component C1q and Fc Receptors

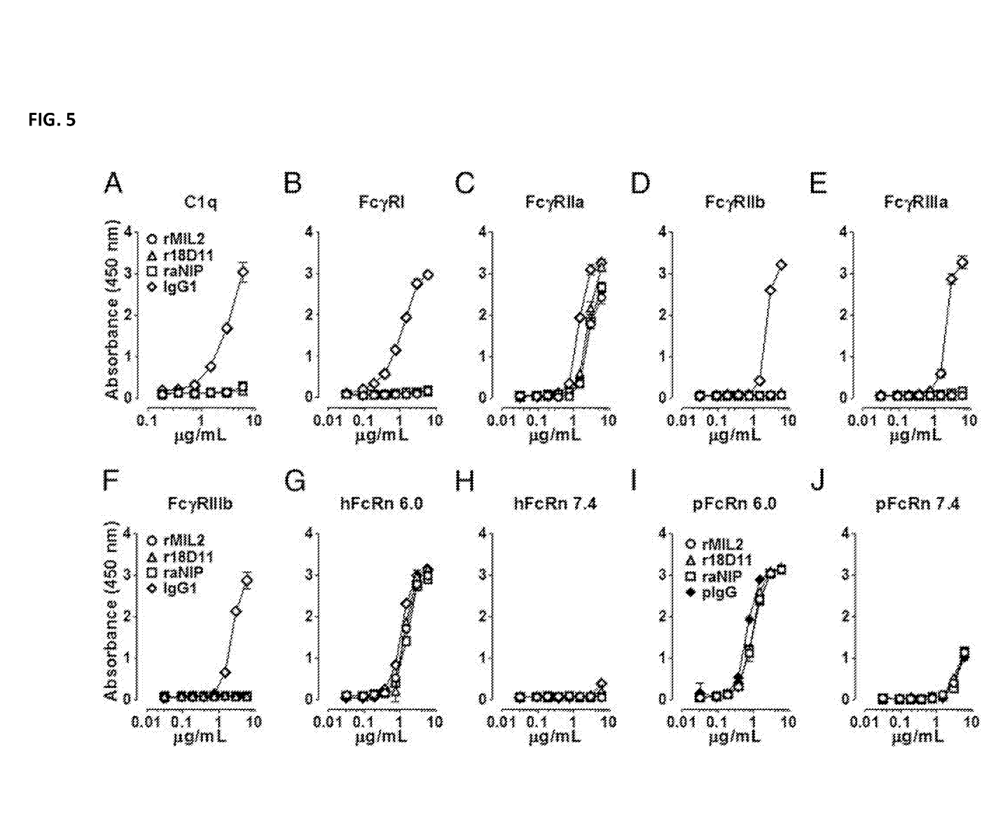

To test further for potential activation of the classical complement pathway and FcR, binding of the recombinant IgG2/4 Abs to C1q and human FcgRs was measured by ELISA (FIG. 5). Importantly, none of them bound to C1q, whereas the positive control, a human IgG1 (rituximab), did so in a dose-dependent manner (FIG. 5A). The same was observed for all tested FcgRs, except for FcgRIIa allotype His131 (FIG. 5C). Here, the recombinant Abs bound to the receptor, though less than human IgG1. Furthermore, binding to the FcRn, which plays a crucial role in IgG t1/2 regulation and biodistribution, was examined. Recombinant IgG2/4 Abs bound both human and porcine FcRn receptors in vitro with dose responses comparable to those for the positive controls, human IgG1, or a porcine IgG pool (FIGS. 5G-K). In accordance with the reported pH dependency, both the human and porcine FcRn bound their IgG ligands at an acidic pH (pH 6.0), whereas binding of IgG at physiologic pH (˜pH 7.4) was negligible. Binding occurs at the CH2 and CH3 domains of the Fc region, with amino acid 435 (His435 in IgG2) being a key contact residue (Roopenian, D. C., and S. Akilesh. 2007. Nat. Rev. Immunol. 7: 715-725).

In Vivo Adverse Effects Induced by Anti-Porcine CD14 Ab Mil2 are not Observed after rMil2 Injection in a Pig

Intravenous bolus injections of Mil2 to pigs have been observed to disturb the porcine hemodynamics by causing severe peripheral vasodilatation, increase in heart rate, drop in systemic arterial pressure and loss of platelets, together interpreted as reactions that appeared to be anaphylaxis. Mil2 and rMil2 were therefore compared for induction of these adverse effects in vivo using Norwegian domestic piglets (FIG. 6A-D). In two piglets, a total of 5.36 mg/kg Mil2 or rMil2 were injected as increasing dose over a period of 45 min (FIG. 6A). In vivo binding of Mil2 and rMil2 to porcine CD14 was demonstrated by the blocked binding of FITC-conjugated Mil2 to CD14-positive granulocytes in blood samples collected during infusion. FITC-conjugated Mil2 was added to the blood samples immediately preceding flow cytometry analyses (FIG. 6B). At a total dose of 1.12 mg/kg, which was reached after 20 min, more than 50% of the available cell-bound CD14 was saturated. Hemodynamic readouts were recorded. Injection of Mil2, but not rMil2, caused an increase in heart rate after 10 min, at which time a total of 0.32 mg/kg Ab had been given (FIG. 6C). For Mil2, the heart rate reached its maximum of ˜300 beats/min during the next 5 min and then slowly fell to baseline. Mil2 injection also caused a reversible drop in mean arterial blood pressure, which again was not seen for rMil2. Finally, Mil2 injection induced a gradual depletion of platelets, whereas rMil2 did not affect platelet counts (FIG. 6D). The loss of free platelets in the presence of Mil2 were also visualized on blood slides from the same blood samples (not shown). Thus, none of the adverse effects observed in vivo with Mil2 were observed with rMil2.

In Vivo Cytokine Response Induced by E. coli was Abolished by rMil2

The biologic effect of rMil2 was then investigated. rMil2 was given as a bolus dose to a piglet, and the leukocyte expression of CD14 before and after this bolus was measured using fluorescence-labeled rMIL in flow cytometry. A reduction in CD14 expression by 94% was observed after the bolus of rMIL2 was given, consistent with a virtually complete saturation of CD14 by rMIL2 in vivo (FIG. 6E). Furthermore, rMil2 virtually abolished the E. coli-induced cytokine response (FIG. 6F-I). TNF, IL-1b, IL-6, and IL-8 were reduced by 71%, 89%, 88%, and 100%, respectively (area under the curve).

In this study, a recombinant anti-porcine CD14 IgG2/4 Ab (rMil2), which showed to be functional with respect to neutralization of LPS-induced cytokine production and free of undesired Fc-mediated effects was generated and characterized. The data demonstrated that rMil2 can be used for in vivo therapeutic intervention of inflammation.

In the current study, a recombinant anti-human CD14 IgG2/4 Ab (r18D11) that blocked CD14-mediated inflammatory responses in a human whole blood model of inflammation, was virtually inert with respect to Fc-mediated binding to complement and FcgRs, and induced no oxidative burst (FIGS. 4 and 5) was generated. Thus, r18D11 finds use in anti inflammatory drug engineering and therapeutic intervention.

To study the many roles of CD14 in vivo, pigs are emerging as a valuable test model system. A recombinant antiporcine CD14 IgG2/4 Ab-rMil2 was generated. The original clone Mil2 from which rMil2 is derived has already been used as intervention in porcine sepsis (Thorgersen, et al. 2010. FASEB J. 24: 712-722). Despite the fact that the application of Mil2 was efficient in reducing the inflammatory response, its bolus application was hampered by the induction of an initial reaction appearing to be anaphylaxis and had a clear limitation for further study. In this study, it was shown that Mil2 induces unwanted IL-8 release in vitro (FIG. 2) and platelet activation both in vitro and in vivo (FIGS. 2 and 6). The latter was accompanied with hemodynamic changes, including decreased arterial blood pressure and increased heart rate. It was demonstrated that none of these effects were induced when rMil2 was used instead, indicating a major step forward with respect to CD14 inhibition. Finally, it was shown that the biologic activity of rMil2 was preserved, as compared with the original Mil2, by blocking leukocyte CD14 and by abolishing E. coli-induced cytokine production in vivo. Mil2 does not affect E. coli survival in whole pig blood, in contrast to a complement inhibitor, that increased bacterial survival (Thorgersen, et al., 2009. Infect. Immun. 77: 725-732).

IgG infusion-related in vivo reactions that appear to be anaphylaxis, as well as FcgR-Ab-dependent cell cytotoxicity or complement-dependent mediated cytotoxicity, are unwanted events in anti-CD14 based inflammatory therapeutic strategy, where maintenance of homeostasis is the main concern. Now, recombinant anti-CD14 IgG2/4 Abs with minimum Fc-mediated effector functions are available. Of the human IgG subclasses, IgG2 and IgG4 exert the least FcgR binding and complement fixation activities, respectively (Bruhns, et al., 2009. Blood 113: 3716-3725; Hamilton, R. G. 1987. Clin. Chem. 33: 1707-1725; Schroeder, et al., 2010. J. Allergy Clin. Immunol. 125(2, Suppl 2)S41-S52). The combination of the two subclasses in a human IgG2/4 subclass hybrid abolished binding to complement and any all FcgRs, except FcgRIIa (allotype His131), a low-affinity activating FcgR (FIG. 5). All conventional FcgRs bind their Fc ligands at a site that involves the lower hinge region and the two CH2 domains (Sondermann, et al., 2001. J. Mol. Biol. 309: 737-749; Ramsland, et al., 2011. J. Immunol. 187: 3208-3217). The recombinant IgG2/4 CH hybrid Abs carry sequences from the human IgG2 subclass in the lower hinge, and all FcgR contact residues in the CH2 domain that were derived from IgG4 are identical to those found in IgG2 (Ramsland et al., supra). Thus, the data are consistent with the fact that FcgRIIa (allotype His131) is the only FcgR that binds IgG2, and thus the IgG2/4 subclass hybrid, with reasonable affinity (Bruhns, et al., 2009. Blood 113: 3716-3725). Importantly, pigs are not known to express FcgRIIa, and the homology between other human and pig FcgRs is more than 60% (Halloran, et al., 1994. J. Immunol. 153: 2631-2641; Qiao, et al., 2006. Vet. Immunol. Immunopathol. 114: 178-184; Zhang, et al., 2006. Immunogenetics 58: 845-849). For example, the most important residues in FcgRIIIa for IgG binding, Trp87 and Trp110, are conserved between the pig and human receptors. FcgRIII has been shown to play a key role in IgG mediated anaphylaxis (Khodoun, et al., 2011. Proc. Natl. Acad. Sci. USA 108: 12413-12418), but is bound only weakly by human IgG2 (Bruhns, et al., 2009. Blood 113: 3716-3725). It is, therefore, not surprising that rMil2 with its IgG2/4 hybrid C region does not induce an anaphylactic reaction in pigs. Human IgG is both readily bound and taken up by pig cells expressing porcine FcRn (Stirling, et al., 2005. Immunology 114: 542-553). FcRn regulates the serum t1/2 of Abs by a recycling mechanism that requires pH dependent binding (Vaughn, D. E., and P. J. Bjorkman. 1998. Structure 6: 63-73). In this study, it was demonstrated that human IgG2/4 subclass hybrid Abs bind porcine FcRn in such a pH-dependent manner.

The monoclonal antibodies described herein are contemplated ti bind to either the N-terminal LPS-binding pocket of CD14 or parts of the LPS-signaling motif. the hydrophobic binding pocket can also accommodate other acylated endogenous and exogenous ligands of CD14 and TLRs (Kim, et al., 2005. J. Biol. Chem. 280: 11347-11351; Albright, et al., Biochem. Biophys. Res. Commun. 368: 231-237; Kelley, et al., 2013. J. Immunol. 190: 1304-1311). Therefore anti-CD14 Abs, like r18D11 and rMil2, may affect pattern recognition signaling upon a wide range of threats, being more efficient than, for example, LPS mimics.

Recently, intensive cross talk has been described for TLR signaling and the complement system, which itself is associated with a plethora of acute and chronic inflammatory disorders (Kohl, J. 2006. Adv. Exp. Med. Biol. 586: 71-94; Ricklin, et al., 2010. Nat. Immunol. 11: 785-797). This functional interplay has been recognized as an important regulatory mechanism to control both innate and adaptive immune responses (Hawlisch, H., and J. Kohl. 2006. Mol. Immunol. 43: 13-21; Hajishengallis, G., and J. D. Lambris. 2010. Trends Immunol. 31: 154-163; Song, W. C. 2012. Toxicol. Pathol. 40: 174-182), and combined inhibition of CD14 and complement has been described as an effective therapeutic approach in conditions associated with detrimental activation of the innate immune system (Mollnes, et al., 2008. Adv. Exp. Med. Biol. 632: 253-263; Barratt-Due, et al., 2012. Immunobiology 217: 1047-1056). It was recently shown that combined inhibition on CD14, using the original clone Mil2, and the complement inhibitor OmCI, reduces inflammation, hemostatic disturbances and improved hemodynamics in a porcine model of E. coli sepsis (Barratt-Due, et al., 2013. J. Immunol. 191: 819-827). These results were obtained despite the adverse effects seen with the original clone Mil2. Experiments described herein demonstrate that rMil2 combined with the complement C5 inhibitor OmCI efficiently attenuates the E. coli-induced cytokine response and TF expression in porcine whole blood without any adverse effects (FIG. 3).

TABLE 1

| Antibody | Chain | Amino acid sequence (N→C) | IMGT¹ |
|---|---|---|---|
| r18D11 | Variable light | MGWSCIILPLVATATGVHGNIVLTQSPASLAVSLGQRATLSCR | IGKV3-10*01 |
| | | ASESVLSYGNSPMHWYQQKPGQPPKLLIYLASNLESGVPAEFG | IGKJ2*01 |
| | | DGGSRTDFTLTIDPVEAGDVATYYCQQNNGDPYTFGGGTKLEI | |
| | | I(R) | |
| | Variable heavy | MGWSCIILFLVATATGVHSEVQLVESGGGLMQPKGSLKLSCAA | IGHV10-1*02 |
| | | SGPTFKTYALWWVRQAPGTGLEWVARIESKSNNYTTYYADGVK | IGHJ3 |
| | | DRFTISRDDSQNMLYLQMNNLKTEDTAMYYCVRPQSGTSFAYW | IGHD2-11*01 |
| | | GQGTLVTVSA(A) | |
| rMIL2 | Variable light | MGWSCIILFLVATATGVHGDIVMTQSQKPMGTSVGDRVSVTCK | IGKV6-15*01 |
| | | ASQYVGTNVAWYQDKPGQSPKALIQSASTRCSGVPDRFTGSGS | IGKJ5*01 |
| | | GTDFTLTLSNVQGKSLADYFCQQYNTYVTPGGGTKLELK(R) | |
| | Variable heavy | MGWSCIILFLVATATGVHSQVELQQPGAELVRPGASVKLSCKA | IGHV1-74*01 or |
| | | SGYTFTTYWMMWVKQRDEDGLEWIGRIDDYDSETHYNQNFKDK | IGHV1-74*04 |
| | | AILTVDKSSSTAYMQLESLTYEDSAYYCTRKRGRQWGAYFDY | IGHJ2*01 |
| | | WGQGTTLTVSS(*A) | IGHD6-2*01 |
| All | Constant light | TVAAPSVFIPDPSDEQLKSGTASVVCLLNNFYPREAKVQWKVD | J00241 |
| | kappa | NALQSGNSQESVTEQDSKDSTYSLSGTLTLGKADYEKHKVYAC | |
| | | EVTHQGLGSPVTKGFNRGEC- | |
| | Constant heavy | STKGPSVFPLAPCSRSTSESTAALGCLVKDYFPEPVTVAKNSG | J00230/ |
| | IgG2/IgG4 | ALTSGVHTPPAVLQSGLYSLSSVVTVPSSNPGTQFYTCNVDH | K01316 |
| | | KPSNTKVDKTVEEICCVECPPCPAPPVAGPSVFLF/ | |
| | | PPKPKDTLMISRTDEVTCVVVDVSQEDPEVQFNGYVDGVEVHN | |
| | | AETKPREEQFNSTTRVVSVLTVLHQDWLNGKEYKCKVSNKGLP | |
| | | SSIEKTIGKAKGQPREPQVYTLPPSQKEMTKNQVSLTCLVEGF | |
| | | YPSDIAVEWESNSQPENNYKTTPPVLDSDGSFFLYSRLTVDKS | |
| | | RWQEGNVFSCSVMHEALHNHYTQKSLSLSLGK- | |

¹IMGT accession number. Nucleotide sequence alignment with the IMGT database revealed at least 90% identity of the variable genes with the listed entries.
[ . . . ], amino acid derived from splicing site, connecting coding sequences of the variable and constant genes in light chains (R, arginine) and heavy chains (A, alanine).
Underlined, leader sequence encoded by the expression vectors; bold underlined, residues PSVFLF are identical in IgG2 and IgG4.

TABLE 2
Primers used for V-gene retrieval

| Binding site | Primer ID | Primer sequence (5′→3′) |
|---|---|---|
| Murine constant heavy γ1* | IgG1CH1_rev | GTTTGCAGCAGATCCAGGG |
| | IgG1CH1_MluI_rev2 | ATACGCGTGTTTGCAGCAGATCCAGGG |
| Murine constant heavy γ2b* | IgG2BCH1_rev | GAGTTCCAAGTCACAGTCACTG |
| | IgG2bCH1_MluI_rev | ATACGCGTAGTTGTATCTCCACACCCAGG |
| Murine constant light kappa* | IgGKC_rev | GCCATCAATCTTCCACTTGACA |
| | IgGKC_MluI_rev | ATACGCGTACTGAGGCACCTCCAGATG |
| Poly-dC tail | PolyG_BglII_fwrd | ATATAGATCTGGGGGGGGGGGGGGGG |

*reverse complementary binding
Underlined, restriction sites for MluI and BglII, respectively

TABLE 3
Synthetic inhibitions of complement correction

| Name | Compound Identity | MW | Structural [symbol] | Size of action | IC₅₀ (μM) | Reference |
|---|---|---|---|---|---|---|
| | Peptide analogues | | C5a | C5aR antagonist | 2 | [symbol] Ovetran et al., 1992 |
| | and derivatives | | | | | |
| | Peptide | | C5a, C-terminal | C5aR antagonist | | Kawai et al., 1991; Kawai et al., 1992 |
| | | | ociapeptide | | | |
| | Peptide | | C5a, Hi [symbol] ¹⁷-modified | C5aR antagonist | | Or et al., 1992 |
| | | | C-terminal ociapeptide | | | |
| | | | analogues | | | |
| | Peptide | | C5a | C5aR antagonist | | Zhang et al., 1997 |
| CD89 | Peptide with [symbol] | | C5a hexapeptide | C5aR antagonist | 0.070 | Konbestis et al., 1991 |
| | substitutions | | | | | |
| PR226 | Peptide | 2137 | C5aR | C5aR antagonist | | Barantl et al., 1992 |
| | | | | at >300 uM | | |
| | | | | agonist at <2.0 uM | | |
| | Peptide | | C5a C-terminus | C5aR antagonist | | Kret [symbol] et al., 1992 |
| | Peptide | 1430 | C [symbol] -tested phage | C3 | Classical [symbol] | Salus et al., 1996 |
| | | | display screening | | alternative: 12 | |
| | Peptide | | CH₂ demain at | | | Roavkk et al., 1979, Lakes et al., 1981 |
| | | | human IgG | | | |
| | Peptide | | C1q | | | Raid et al., 1977 |
| | Peptide containing | | | C3 convertase | | K [symbol] et al., 1977 |
| | Phe/Tye | | | | | |
| | Peptide | | Factor D-related | Factor D, alternative | | Lowavers et al., 1982 |
| | | | humapeptides | | | |
| CEP2 | Peptide | | C1q B chain | C1q | | Pryal et al., 1997 |
| | | | helical region | | | |
| | Peptide | | C3 | C3 | 25 | Ogets and Low, 1997 |
| DEP | Diisopropyl | | | factor D | | Cola et al., 1997; Festure et al., 1974 |
| | Humanphosphate | | | | | |
| | (model compound) | | | | | |
| BCX-1470 | | | | factor D | 0.096 | Kilpoiriek, 0997* |
| | K-76 analizes | | K-76 (see table 6) | | Classical 1,600; | Kautman et al., 199 [symbol] |
| | | | | | alternative: 2,600 | |
| TKIXc | K-76 derivative | | K-76 (see table 6) | | Classical 100; | Sindelar et al., 1996 |
| | | | | | alternative: 1,900 | |
| K-75 COOH | K-76 derivative | | K-76 (see table 6) | | C [symbol] | Huag et al., 1979; Miyakaki et al., |
| | | | | | | 1984; Tanaks et al., 1996 |
| FUT-175 | [symbol] | | | Classical, alternative | | Fujii and Hibecus, 1981; Hitonei and |
| | | | | | | Fujii, 1992, Iksri et al., 1982; |
| | | | | | | Angama et al., 1984; Excli et al., |
| | | | | | | 1980; Teackats et al., 1990; |
| | | | | | | HU [symbol] et al., 1992, Issue et al., |
| | | | | | | 1997 |
| PS-oligo | Oliged [symbol] | | | Classical, alternative | | Share et al., 1997 |
| | containing phosphar [symbol] | | | | | |
| | [symbol] linkages | | | | | |
| | Other compounds | | | | | Fujii and Angans, 1994; Angar, 1994 |

*Kilpanik, JM Development of [symbol] [symbol] of factor D [symbol] [symbol]
indicates data missing or illegible when filed

As shown in Table 4 below, r18D11 has a variable light chain amino acid sequence described by SEQ ID NO:1 and a variable heavy chain amino acid sequence described by SEQ ID NO:2. rMIL2 has a variable light chain amino acid sequence described by SEQ ID NO:3 and a variable heavy chain amino acid sequence described by SEQ ID NO:4. In some embodiments, antibodies have a constant light chain kappa amino acid sequence described by SEQ ID NO:5 and a constant heavy chain (IgG2/IgG4) amino acid sequence described by SEQ ID NO:6.

TABLE 4

| Antibody | Chain | Amino acid sequence (N→C) | IMGT¹ |
|---|---|---|---|
| r18D11 | Variable light | MGWSCIILPLVATATGVHGNIVLTQSPASLAVSLGQRATLSCR | IGKV3-10*01 |
| | | ASESVLSYGNSPMHWYQQKPGQPPKLLIYLASNLESGVPAEFG | IGKJ2*01 |
| | | DGGSRTDFTLTIDPVEAGDVATYYCQQNNGDPYTFGGGTKLEI | |
| | | I(R) | |
| | Variable heavy | MGWSCIILFLVATATGVHSEVQLVESGGGLMQPKGSLKLSCAA | IGHV10-1*02 |
| | | SGPTFKTYALWWVRQAPGTGLEWVARIESKSNNYTTYYADGVK | IGHJ3 |
| | | DRFTISRDDSQNMLYLQMNNLKTEDTAMYYCVRPQSGTSFAYW | IGHD2-11*01 |
| | | GQGTLVTVSA(A) | |
| rMIL2 | Variable light | MGWSCIILFLVATATGVHGDIVMTQSQKPMGTSVGDRVSVTCK | IGKV6-15*01 |
| | | ASQYVGTNVAWYQDKPGQSPKALIQSASTRCSGVPDRFTGSGS | IGKJ5*01 |
| | | GTDFTLTLSNVQGKSLADYFCQQYNTYVTPGGGTKLELK(R) | |
| | Variable heavy | MGWSCIILFLVATATGVHSQVELQQPGAELVRPGASVKLSCKA | IGHV1-74*01 or |
| | | SGYTFTTYWMMWVKQRDEDGLEWIGRIDDYDSETHYNQNFKDK | IGHV1-74*04 |
| | | AILTVDKSSSTAYMQLESLTYEDSAYYCTRKRGRQWGAYFDY | IGHJ2*01 |
| | | WGQGTTLTVSS(*A) | IGHD6-2*01 |
| All | Constant light | TVAAPSVFIPDPSDEQLKSGTASVVCLLNNFYPREAKVQWKVD | J00241 |
| | kappa | NALQSGNSQESVTEQDSKDSTYSLSGTLTLGKADYEKHKVYAC | |
| | | EVTHQGLGSPVTKGFNRGEC- | |
| | Constant heavy | STKGPSVFPLAPCSRSTSESTAALGCLVKDYFPEPVTVAKNSG | J00230/ |
| | IgG2/IgG4 | ALTSGVHTPPAVLQSGLYSLSSVVTVPSSNPGTQFYTCNVDH | K01316 |
| | | KPSNTKVDKTVEEICCVECPPCPAPPVAGPSVFLF/ | |
| | | PPKPKDTLMISRTDEVTCVVVDVSQEDPEVQFNGYVDGVEVHN | |
| | | AETKPREEQFNSTTRVVSVLTVLHQDWLNGKEYKCKVSNKGLP | |
| | | SSIEKTIGKAKGQPREPQVYTLPPSQKEMTKNQVSLTCLVEGF | |
| | | YPSDIAVEWESNSQPENNYKTTPPVLDSDGSFFLYSRLTVDKS | |
| | | RWQEGNVFSCSVMHEALHNHYTQKSLSLSLGK- | |

¹IMGT accession number. Nucleotide sequence alignment with the IMGT database revealed at least 90% identity of the variable genes with the listed entries.
[ . . . ], amino acid derived from splicing site, connecting coding sequences of the variable and constant genes in light chains (R, arginine) and heavy chains (A, alanine).
Underlined, leader sequence encoded by the expression vectors; bold underlined, residues PSVFLF are identical in IgG2 and IgG4.

TABLE 5
Primers used for V-gene retrieval

| Binding site | Primer ID | Primer sequence (5′→3′) |
|---|---|---|
| Murine constant heavy γ1* | IgG1CH1_rev | GTTTGCAGCAGATCCAGGG |
| | IgG1CH1_MluI_rev2 | ATACGCGTGTTTGCAGCAGATCCAGGG |
| Murine constant heavy γ2b* | IgG2BCH1_rev | GAGTTCCAAGTCACAGTCACTG |
| | IgG2bCH1_MluI_rev | ATACGCGTAGTTGTATCTCCACACCCAGG |
| Murine constant light kappa* | IgGKC_rev | GCCATCAATCTTCCACTTGACA |
| | IgGKC_MluI_rev | ATACGCGTACTGAGGCACCTCCAGATG |
| Poly-dC tail | PolyG_BglII_fwrd | ATATAGATCTGGGGGGGGGGGGGGGG |

*reverse complementary binding
Underlined, restriction sites for MluI and BglII, respectively

1. An isolated chimeric mouse human monoclonal antibody that binds to CD14, wherein said antibody has a variable light chain amino acid sequence selected from SEQ ID NO: 1 and sequences that are least 80% identical to SEQ ID NO:1 and a variable heavy chain amino acid sequence selected from SEQ ID NO: 2 and sequences that are least 80% identical to SEQ ID NO:2.

2. An isolated chimeric mouse human monoclonal antibody that binds to CD14, wherein said antibody has a variable light chain amino acid sequence selected from SEQ ID NO: 3 and sequences that are least 80% identical to SEQ ID NO:3 and a variable heavy chain amino acid sequence selected from SEQ ID NO: 4 and sequences that are least 80% identical to SEQ ID NO:4.

3. The antibody of claim 1, wherein said antibody comprises a human IgG2/IgG4 hybrid C region.

4. The antibody of claim 3, wherein said antibody has a constant light chain amino acid sequence selected from SEQ ID NO: 5 and sequences that are least 80% identical to SEQ ID NO:5 and a constant heavy chain amino acid sequence selected from SEQ ID NO: 6 and sequences that are least 80% identical to SEQ ID NO:6.

5. The antibody of claim 1, wherein said antibody is an antibody fragment.

6. The antibody of claim 5, wherein said fragment is selected from Fab, Fab′, Fab′-SH, F(ab′)₂, Fv, and scFv variants.

7. The antibody of claim 1, wherein said antibody is a full length antibody.

8. The antibody of claim 1, wherein said antibody comprises an antibody fragment fused to a non-antibody molecule.

9. The antibody of claim 8, wherein said non-antibody molecule is a human serum albumin polypeptide.

10. The antibody of claim 9, wherein said human serum albumin polypeptide is a variant polypeptide.

11. The antibody of claim 1, wherein said antibody is a humanized antibody.

12. The antibody of claim 1, wherein said antibody inhibits at least one biological activity of CD14.

13. The antibody of claim 1, wherein said antibody does not induce Fc-mediated side effects.

14. A pharmaceutical composition comprising the antibody of claim 1.

15. The pharmaceutical composition of claim 14, wherein said pharmaceutical composition further comprises an inhibitor of a complement component.

16. The pharmaceutical composition of claim 15, wherein said complement component is C5.

17. The pharmaceutical composition of claim 14, wherein said inhibitor is selected from eculizumab, OmCI, and those shown in Table 3.

18. A method of treating or preventing sepsis, comprising:
administering the pharmaceutical composition of claim 14 to a subject diagnosed with or at risk of sepsis.

19-20. (canceled)
